(19) United States
(12) Patent Application Publication
(10) Pub. No.: US 2018/0055737 A1
(43) Pub. Date: Mar. 1, 2018

(54) Overdose Protecting Dispensing Device

(71) Applicant: Stephanie Figueroa, New Braunfels, TX (US)

(72) Inventor: Stephanie Figueroa, New Braunfels, TX (US)

(21) Appl. No.: 15/247,214

(22) Filed: Aug. 25, 2016

(51) Int. Cl. A61J 7/04; B65D 83/00; A61J 7/00

(52) U.S. Cl. CPC A61J 7/04; A61J 7/0481; A61J 7/0053; B65D 83/0033

(57) ABSTRACT

An improved syringe is disclosed. One improved feature is the addition of a timing unit that can be programmed to give the user audio and visual alarm at the end of a preset time countdown. The timing unit can be reversibly attached to a syringe, in some embodiments. This allows the user to quickly and easily swap out the syringe attached to the timing unit. In other embodiments the timing unit is integrated directly onto the syringe. An internal memory unit stores the last time set, allowing for quick repeats of the same time interval. The memory and display also allow the user to enter or select the medicine being taken to prevent confusion. The advantage of this invention is in the increased safety and efficacy of taking liquid medicines.

### CROSS-REFERENCE TO RELATED APPLICATIONS

This application does not claim the benefit of provisional patent.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The vast majority of prescribed and over the counter medicines require the consumer to take the medicine according to a doctor's prescription or manufacturer's recommended protocol. The average person is familiar with instructions to take a predetermined amount of medicine at some regular interval of hours. The reason for this is that the efficacy of the treatment may be affected greatly by the adherence to that dosing schedule. Beyond efficacy, however there is also danger in the improper consumption of medicines which require a scheduled dosing. Though simple in theory, there are many possible factors that may contribute to errors or failures to adhere to the dosing schedule. For example, a person may find it more difficult to remember to self-administer a particular medicine when they are on a complex combination of multiple medicines. Those medicines may have unique doses and intervals that are difficult to keep track of simultaneously. Other people may be taking medicines for illnesses that affect mental acuity or awareness. Other factors include daily distractions such as work or familial responsibilities. Another common scenario is the parent caring for multiple children. Multiple children with illness can cause a scheduling complication because children's medicine dosages are often based on age and weight which are likely to be different among siblings. In those situations, it would again be difficult to maintain the correct schedule. Poison Control, a national capital poison center with website www.poison.org, using US national data reported that for 2014, an incredible 12.6% of poison exposures across all age groups resulted from Therapeutic Error.

Prior patents have been granted for inventions seeking to solve this problem by attaching a basic timing device to a medicine bottle. These represent a particular type of solution, though an imperfect one. Despite the numerous granted patents, the lack of availability to purchase such devices in the common marketplaces where one would expect to find such items—for example at a local pharmacy and convenience store—is telling of their perceived failure by the market.

Others have taken a strategy whereby a box with compartments is fitted with timing devices. Some even automate the dispensing of medicine by unlocking or opening the appropriate box according to the dosing schedule. These too have not met great commercial success. One particular shortcoming is that these are designed for pills, ignoring the substantial percentage of medicines that are dispensed in liquid form via syringes.

### BRIEF SUMMARY OF THE INVENTION

The present invention seeks to overcome the shortcomings mentioned above by providing a novel device for aiding a user to keep up with the dosing schedule for liquid medicines. This device seeks to reduce instances of overdosing, dose-skipping, and medicine confusion. The invention unexpectedly combines a programmable timing device, with an alarm, and a medicine dispensing syringe.

The invention transforms the simple syringe into a smart device that is more useful and safer to use.

### BRIEF DESCRIPTION OF THE SEVERAL VIEWS OF THE DRAWING

FIG. 1 shows an embodiment of the invention having the timing unit attached to the top end of the syringe plunger.

FIG. 2 shows an embodiment having a user interface side of the timing unit, comprised of a start/stop button, a set button, a visual alarm, an audio alarm, and a display screen. The up arrow and down arrow buttons can be seen along the left side wall of the timing unit.

FIG. 3 shows an embodiment having a battery under a battery cover on the wall of the timing unit located opposite the user interface side.

FIG. 4 shows an embodiment having a screw fastened tab style battery cover on the wall of the timing unit located opposite the user interface side.

FIG. 5 shows an embodiment having a threaded ring on the wall of the timing unit located opposite the user interface side.

FIG. 6 shows the embodiment of FIG. 5 having a syringe retainer plate with threaded edge corresponding to the threaded ring on the timing unit.

FIG. 7 shows an embodiment having two strap engagement wells and engagement bars located on the wall of the timing unit located opposite the user interface side.

FIG. 8 shows the embodiment of FIG. 7 having a strap for engaging the engagement wells and engagement bars, and for securing the syringe.

FIG. 9 shows another embodiment that clamps onto the syringe plunger top via a set of tensioned retaining lips (urged together via springs) located on the wall of the timing unit located opposite the user interface side.

FIG. 10 shows an angled view of the embodiment of FIG. 9, where the syringe is shown as it would be positioned to engage the retaining lips located on the wall of the timing unit located opposite the user interface side.

FIG. 11 shows an embodiment from an angled view of the syringe as it would be positioned to engage an adhesive strip placed on the wall of the programmable timing unit located opposite the user interface side.

FIG. 12 shows the flow chart representing the control scheme and configuration of an embodiment of the invention.

FIG. 13 shows the relation of the components of an embodiment of the invention.

### DETAILED DESCRIPTION OF THE INVENTION

A basic syringe is composed of a plunger having a top end, an elongated member, and a plunger tip positioned opposite the top end. The plunger is positioned inside a barrel so that the plunger tip engages the inner wall of the barrel to form a seal. The barrel has a flange at the end positioned closest to the plunger top end. The barrel has a tip located opposite the flange. The barrel tip tapers down to a smaller diameter compared to the barrel. To use the syringe, the plunger tip is first pushed into the barrel so that it is adjacent to the interior of the barrel tip. The barrel tip is then submerged into the target liquid, such as a medicine. The plunger top end is pulled, moving the plunger tip away from the barrel tip. This draws the liquid into a cavity created between the barrel interior, the barrel tip, and the plunger tip. Syringes typically feature convenient unit markings on the outside of the barrel so that the user knows how much liquid has been drawn into the syringe for a given plunger tip position. To dispense the liquid, the plunger top is pushed toward the barrel flange, forcing the liquid out through the barrel tip.

The following descriptions are structured to focus on several preferred embodiments. As is commonly understood, the details of these particular descriptions are intended to be illustrative and should not be construed as limitations on the scope of the invention. One of ordinary skill in the art will appreciate that there are many other possible embodiments based on the disclosures made here which are not expressly discussed in detail.

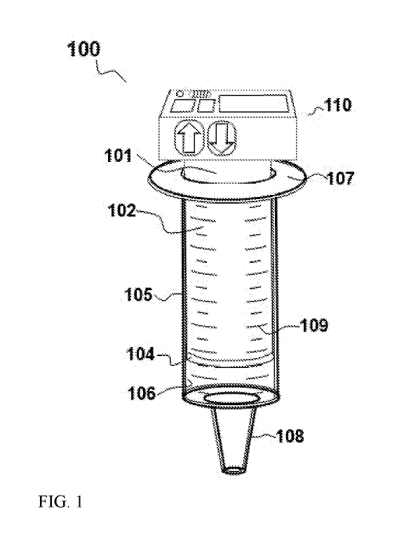

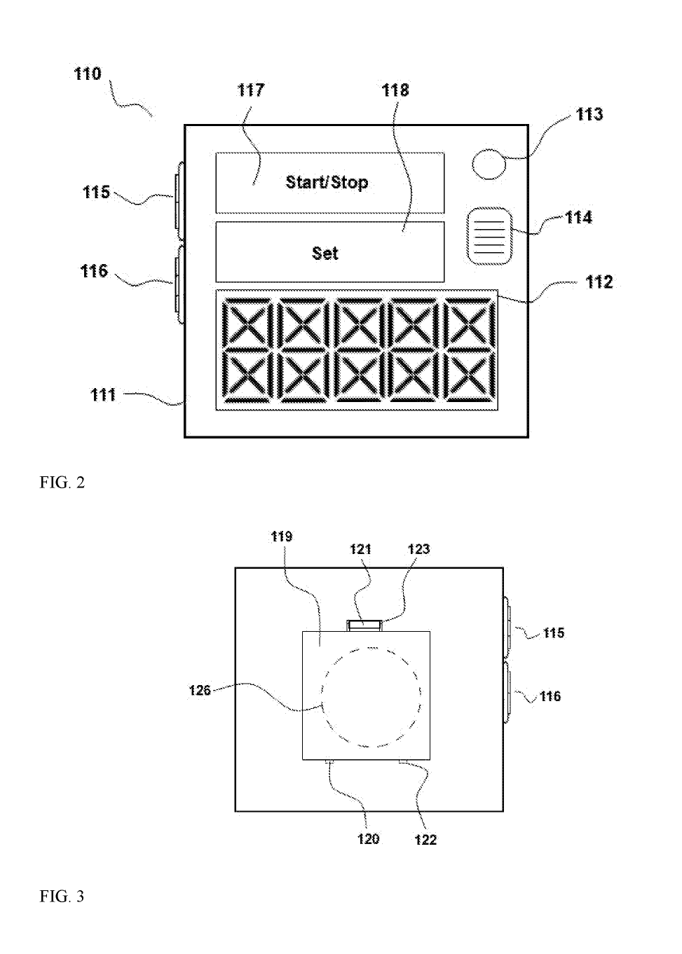

With respect to FIG. 1, syringe 100 is a first embodiment comprised of a plunger 101, a barrel 105, and timing unit 110. The plunger 101 is comprised of an elongated member 102 with two ends. At one end is plunger top end 103, and at the other end is plunger tip 104. Plunger tip 104 is positioned inside of barrel 105 so that it makes contact with barrel inner wall 106. This contact forms a seal that can be moved up and down the length of the barrel 105. Barrel 105 is further comprised of barrel flange 107, positioned at the end closest to the plunger top end 103, when plunger 101 is inserted into barrel 105. Barrel 105 is further comprised of a barrel tip 108, positioned at the end opposite the barrel flange 107. Barrel 105 is further comprised of unit markings 109, positioned on the exterior wall of barrel 105.

Still referring to FIG. 1 and syringe 100, the timing unit 110 is positioned at the plunger top end 103. The means of attachment between the timing unit 110 and the plunger top end 103 will be discussed further in a later paragraph. For now, the focus is on the timing unit 110.

In the embodiment shown in FIG. 1 the timing unit 110 is comprised of an outer casing 111. There is a display 112, a visual alarm indicator 113, an audio alarm indicator 114, a start/stop button 117, and a set button 118 on a first side of the outer casing 111. On another side adjacent to the first side of outer casing 111, there is an up arrow button 115 and a down arrow button 116.

In another embodiment, there may be only a first button for controlling the timing unit 110. In another embodiment there is no visual alarm indicator, such as a light. In that embodiment, the display may be used for visual alarms.

On another side of the outer casing 111 that is opposite the side containing the display 112 there is a battery cover 119. The battery cover 119 is reversibly attached to the outer casing 111. In FIG. 3 there is an embodiment in which the battery cover 119 is comprised of battery cover tabs 120 and battery cover tongue 121. The battery cover 119 is reversibly attached by inserting battery cover tabs 120 into the corresponding casing tab slots 122 formed into outer casing 111, and then urging battery cover tongue 121 into corresponding groove 123 formed into outer casing 111.

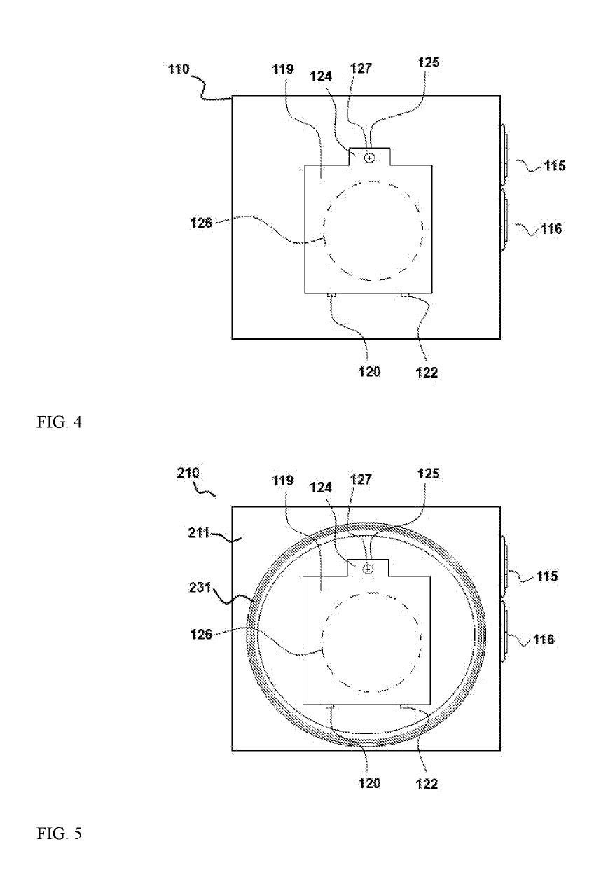

In FIG. 4, an alternative embodiment having battery cover 119 is shown. Instead of battery cover tongue 121 fitting into groove 123, the battery cover 119 is comprised of screw tab 124 which corresponds to screw receiver 125 on outer casing 111. A screw 127 is used to attach screw tab 124 to screw receiver 125.

The FIG. 3 and FIG. 4 embodiments are illustrative of two particular methods of securing battery 126 inside of the outer casing 111 but numerous other methods will be readily known to those having ordinary skill in the art.

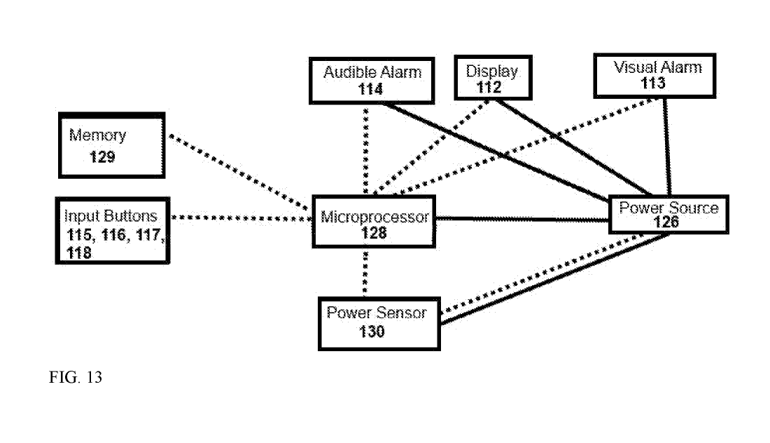

Still referring to syringe 100, timing unit 110 is further comprised of a microprocessor 128, a memory unit 129, and a power sensor 130. For reference see FIG. 13. The power source, here battery 126, powers the microprocessor 128, the display 112, the visual alarm indicator 113, the audio alarm indicator 114, and the power sensor 130. The power sensor 130 is in communication with the power source and relays information about the battery 126 status to the microprocessor 128. The microprocessor 128 is in communication with the memory 129, the display 112, the visual alarm indicator 113, and the audio alarm indicator 114. The microprocessor 128 receives input from the input buttons—here those are the up arrow button 115, the down arrow button 116, the start/stop button 117, and the set button 118. Though it would be readily understood to one having ordinary skill in the art, the visual alarm indicator can be comprised of a light, such as an LED, a color changing area visible through a window on the outer casing, or many other substitutable options.

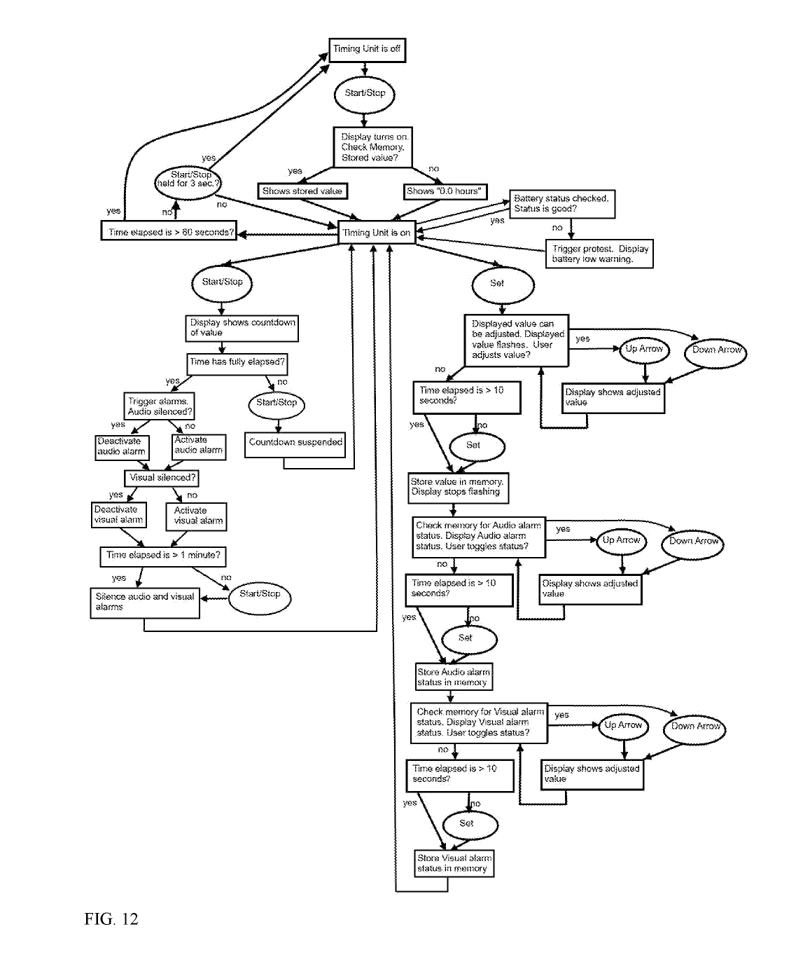

Again referring to syringe 100, timing unit 110 is operated according to the scheme laid out in the flow chart of FIG. 12. The user begins with a timing unit 110 in an off state. Pressing the start/stop button 117 will power on the display 112 and task the microprocessor 128 with checking the memory unit 129 for a stored time value. If there is a stored time value, the display will show it; if there is not a stored value, the display will default to show “0.0 hours.” The timing unit 110 is now in the ready state.

The battery 126 status is checked via power sensor 130. If the status is good, then the unit remains in the ready state; if the status is not good, then a protest is triggered and the display 112 shows a low battery warning, such as “Lo Bat.”

From the ready state, set button 118 can be pressed by the user to enter a program mode. In the program mode, the time value on the display 112 flashes. The user can then use the up arrow button 115 and the down arrow button 116 to adjust the time value. If no adjustment is made for ten seconds, or if the user presses the set button 118, the time value is stored in the memory and the display 112 stops flashing. The microprocessor 128 checks the memory unit 129 for an audio alarm on/off status, and the display 112 shows the current value of that status. The user can use the up arrow button 115 and the down arrow button 116 to toggle the status, and the display 112 shows the changed status. If no change is made for ten seconds, or if the user presses the set button 118, the audio alarm on/off status value is stored in the memory. The microprocessor 128 checks the memory unit 129 for a visual alarm on/off status, and the display 112 shows the current value of that status. The user can use the up arrow button 115 and the down arrow button 116 to toggle the status, and the display 112 shows the changed status. If no change is made for ten seconds, or if the user presses the set button 118, the visual alarm on/off status value is stored in the memory. The timing unit 110 automatically returns to the ready state.

From the ready state, the start/stop button 117 can be pressed by the user to begin the countdown, which is shown on display 112. If the countdown has not fully finished, the user can press the start/stop button 117 to suspend the countdown. The timing unit 110 returns to the ready state with the time value at the moment of suspension shown on display 112. Thus, start/stop button 117 can be pressed to resume the countdown. Alternatively, the user can press the set button 118 to adjust the time value or alarm settings.

If the countdown is started and reaches a “0” time value—in other words, the countdown is fully completed—then the timing unit 110 is in the trigger alarms state. The microprocessor 128 checks the memory unit 129 for the audio alarm on/off status value and proceeds to activate the audio alarm indicator 114 if the alarm status has an “on” status. The microprocessor 128 checks the memory unit 129 for the visual alarm on/off status value and proceeds to activate the visual alarm indicator 113 if the alarm status has an “on” status. If one minute has elapsed in the trigger alarms state—or if the user presses the start/stop button 117—then the timing unit 110 returns to the ready state.

If the timing unit 110 is idle at the ready state for more than 60 seconds—or if the user presses and holds the start/stop button 117 for more than 3 continuous seconds, the timing unit 110 will return to the off state.

The foregoing paragraphs illustrate one embodiment, though other control schemes could be substituted. In an alternative embodiment, the timing unit 110 only has one button, and that single button is used to control the device according to a similar method using short, long holds, and rapid taps.

In another embodiment, not shown in the drawings, while in the program mode the user can manually enter or select from a list the medicine that the syringe is intended to be used with. The entered or selected medicine is shown in the display to prevent confusion on subsequent doses.

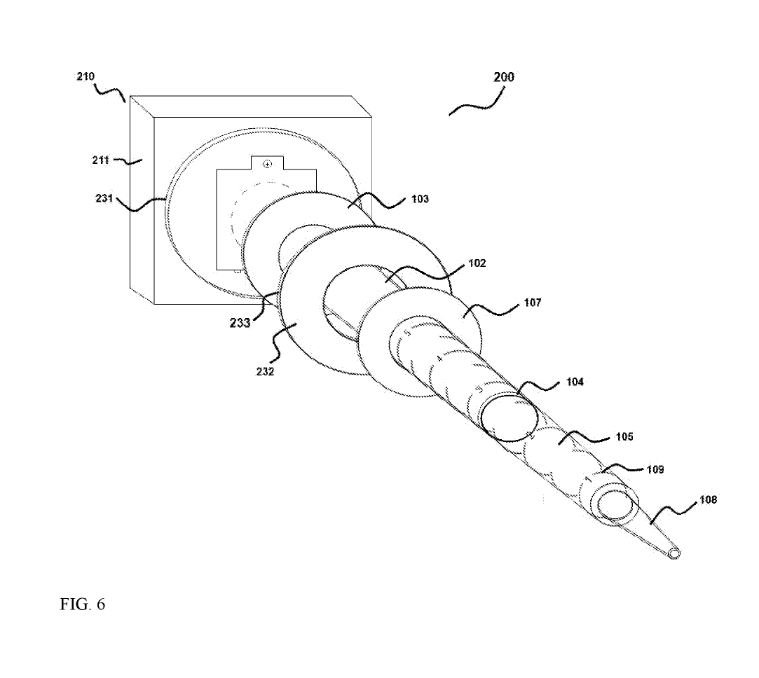

Returning now to the method of attaching the timing unit to the syringe top end, now see FIG. 5 for a first embodiment. In FIG. 5 and FIG. 6, syringe 200 is comprised of timing unit 210 instead of timing unit 110. Timing unit 210 shares many features with timing unit 110, but the outer casing 211 of timing unit 210 is additionally comprised of threaded ring 231. The threaded ring 231 is positioned on the same side as the battery cover 119, and centered around it so that the ring does not interfere with the removal or installation of battery cover 119. The diameter of the threaded ring 231 is larger than the plunger top end 103.

The assembly position of syringe 200 is shown in FIG. 6. First, the plunger 101 is separated from barrel 105. A retainer plate 232, having an outer diameter corresponding to the threaded ring 231, is comprised of a threaded edge 233 and a center hole. The center hole is of a sufficient diameter to accommodate the plunger's 101 elongated member 102. The plunger tip 104 is passed through the center hole of retainer plate 232 so that it lays adjacent to the plunger top end 103. Plunger tip 104 can be reinserted into barrel 105. Retainer plate 232 is then screwed onto the threaded ring 231 of the outer casing 211. Once assembled, the syringe 200 can be used.

In another embodiment not shown in the drawings, magnets could be used as the means for attachment instead of the threaded ring and retainer plate described above.

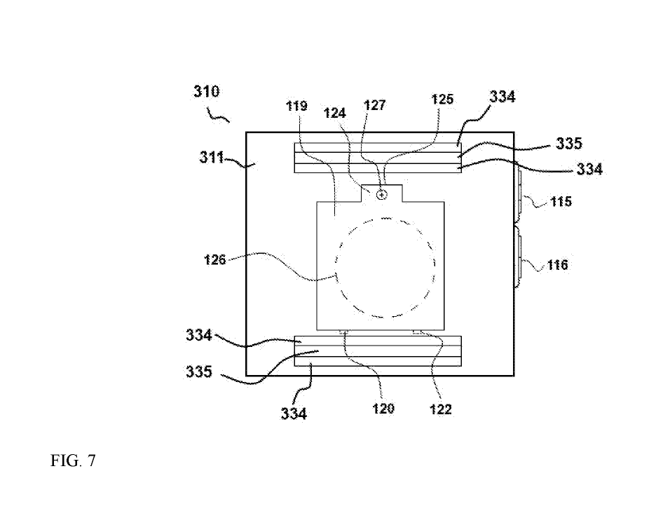

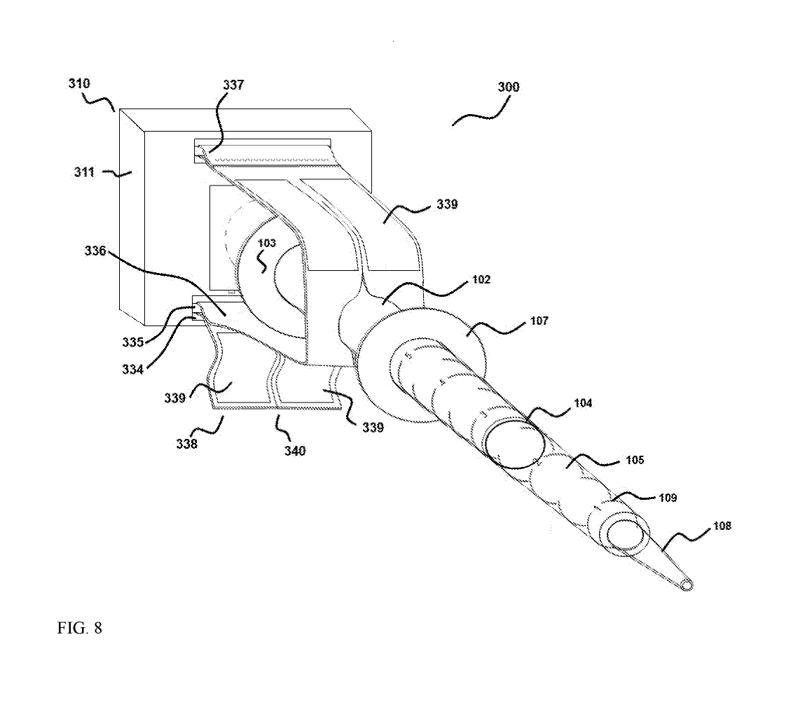

In another embodiment, shown in FIG. 7 and FIG. 8, syringe 300 is comprised of timing unit 310. Timing unit 310 shares many features with timing unit 110, but the outer casing 311 of timing unit 310 is additionally comprised of a pair of strap engagement wells 334 positioned substantially parallel and at opposite ends of the same side of timing unit 310 on which battery cover 119 is located. Timing unit 310 is further comprised of a pair of strap engagement bars 335 positioned across the strap engagement well 334. The strap engagement wells 334 are sufficiently long and wide to allow a strap 336 (seen in FIG. 8) to pass into the engagement well 334, around the engagement bar 335, and back out of the engagement well 334. The engagement bar 335 must be sufficiently sturdy to resist movement away from the engagement well 334 and withstand the tensioning of strap 336.

The assembly position and additional features of syringe 300 are shown in FIG. 8. The strap 336 is comprised of a first end. The first end of strap 336 has a loop 337 formed around one of the engagement bars 335. In FIG. 8, the loop is secured by sewing the first end to the strap at the point where the loop 337 is completed. It is understood that other methods of permanent or reversible attachment could easily be substituted in alternative embodiments. Such methods could include use of magnets, adhesives, melting, rivets, buttons, clasps, hooks, hook-and-loops, etc. The strap 336 is further comprised of a second end, free end 338. The free end 338 is passed into the other engagement well 334, around the engagement bar 335, and back out of the engagement well 334. The free end 338 can be tensioned by the user and then folded back upon strap 336. The free end 338 and the strap 336 can be reversibly attached in this position. Here in FIG. 8, the free end 338 and the strap 336 are in the unattached or open position. In this embodiment, syringe 300, the free end 338 and the strap 336 are attached using opposing sections of hook-and-loop fastener material, referred to as fastener 339. It is understood that other methods of reversible attachment could easily be substituted in alternative embodiments. Such methods could include use of magnets, adhesives, rivets, buttons, clasps, hooks, etc. The strap 336 is further comprised of a strap slit 340 running along the central axis defining the length of the strap. The strap slit begins near the loop 337 and terminates at the free end 338. The strap slit 340 effectively divides the strap 336 into two segments for a large portion of its length.

In another embodiment not shown in the drawings, the strap 336 is constructed of a material with some elasticity. In another embodiment, the strap 336 has a strap slit 340 that terminates prior to reaching the free end 338.

To assemble syringe 300 the strap 336 must be in the unattached or open position. Then, the plunger top end 103 is passed through strap slit 340 at a position along strap 336 that is between the pair of engagement wells 334. The plunger 101 does not need to be separated from barrel 105 to do this. The strap 336 free end 338 is tensioned and folded back to engage the fastener 339. In this position, the syringe top end 103 should be positioned between the strap 336 and the outer casing 311. Once assembled, the syringe 300 can be used.

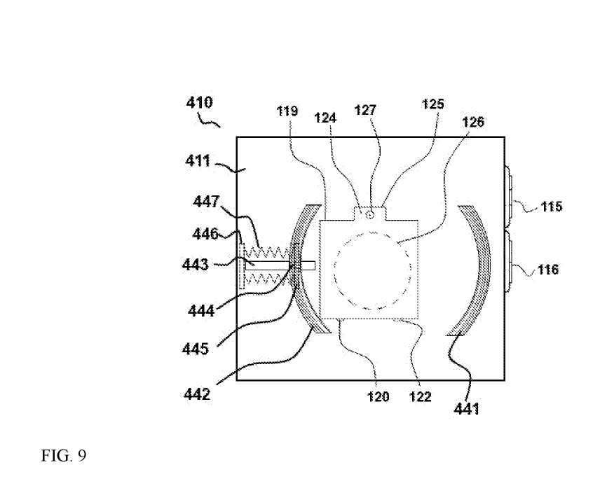

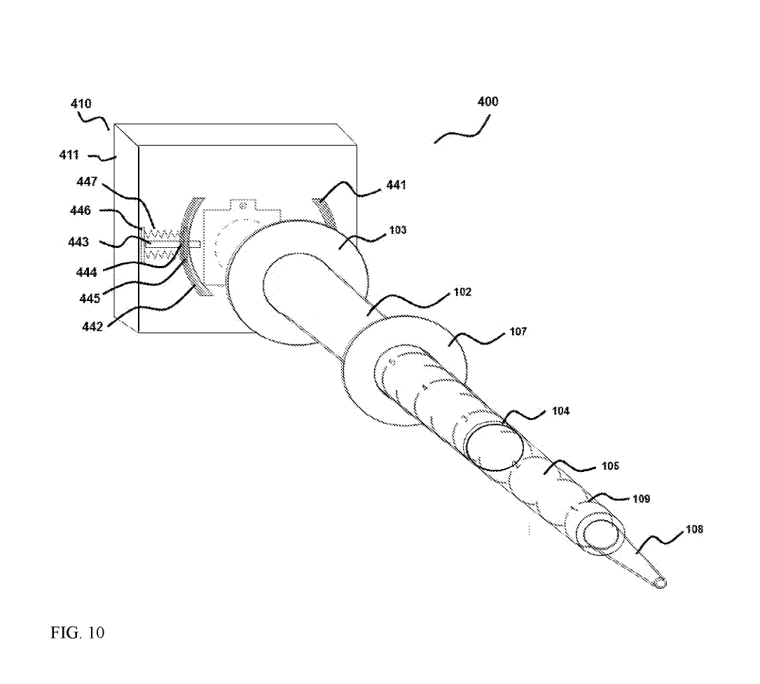

In another embodiment, shown in FIG. 9 and FIG. 10, syringe 400 is comprised of timing unit 410. Timing unit 410 shares many features with timing unit 110, but the outer casing 411 of timing unit 410 is additionally comprised of a stationary retaining lip 441, a tensioned retaining lip 442, a guide well 443, a guide post 444, a load plate 445, a load backstop 446, and springs 447.

The stationary retaining lip 441 is defined by a wall rising perpendicular to the plane defining the side of timing unit 410 on which battery cover 119 is located. The wall of the stationary retaining lip 441 has at least one shoulder portion along the top edge that is wider than the wall, creating the lip. Stationary retaining lip 441 is positioned along one edge of the wall of timing unit 410 upon which it is located. The shape of stationary retaining lip 441 is further defined as an arc of the circle that would sit co-planar to the wall of timing unit 410 upon which it is located.

The tensioned retaining lip 442 has a shape largely defined by the mirror reflection of stationary retaining lip 441. However, tensioned retaining lip 442 differs from stationary lip 441 in that it is attached at its center to guide post 444 which extends perpendicular and away from the plane defining the arc of tensioned retaining lip 442. This guide post 444 is a short member positioned in guide well 443. Guide well 443 is a narrow channel formed into the outer casing 411. Inside the outer casing 411, there are springs 447 trapped under tension between the load backstop 446 and the load plate 445. The tension is created along a plane parallel to the wall of timing unit 410 upon which the guide well 443 is located, and directly beneath the guide well 443. The load plate 445 is positioned interior to and along the wall of outer casing 411 that connects the walls on which the display 112 and the battery cover 119 are located. In other words, the load backstop 446 is along the interior of the wall of outer casing 411 that is on the opposite side of timing unit 410 compared to the wall that forms the edge along which the stationary retaining lip 441 is located. The load plate 445 is attached to the guide post 444. Thus, the tensioned retaining lip 442 is able to slide back and forth along the length of the guide well 443, but due to the tension of the springs 447 is urged toward the end of the guide well 443 that is closest to stationary retaining lip 441.

To assemble syringe 400 the plunger top end 103 is pressed against the concave side of tensioned retaining lip 442 causing it to slide it back far enough to create a distance between the tensioned retaining lip 442 and the stationary retaining lip 441 that is larger than the width, or diameter, of the plunger top end 103. Then the plunger top end 103 is pushed flat against the side of outer casing 411 on which the battery cover 119 is located, maintaining the tension against the tensioned retaining lip 442. Finally, the tension is released causing the tensioned retaining lip 442 and the stationary retaining lip to clamp against the plunger top end 103. The plunger 101 does not need to be separated from barrel 105 to do this. Once assembled, the syringe 400 can be used.

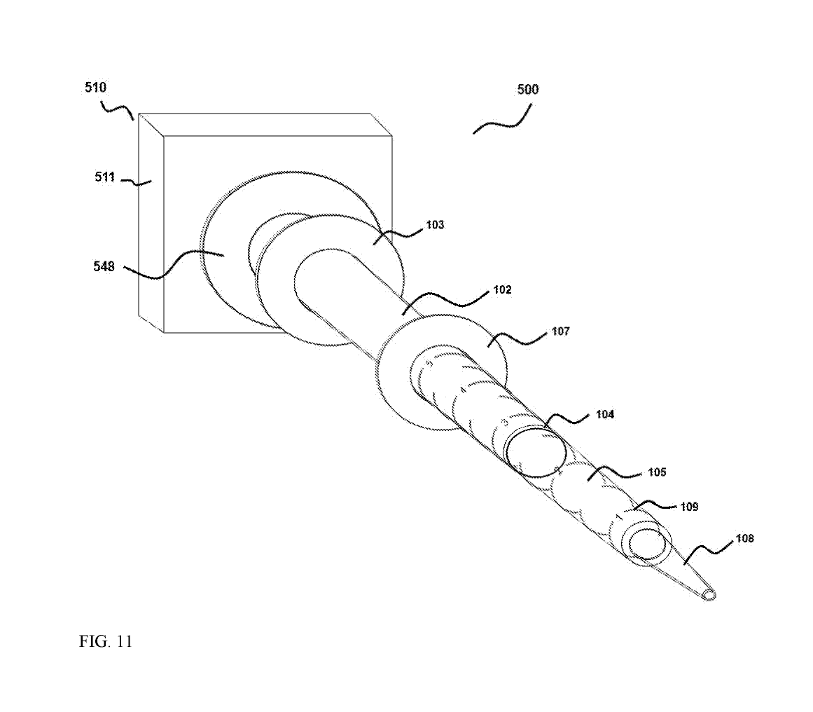

In another embodiment seen in FIG. 11, syringe 500 is depicted. Though syringe 500 shares many features with syringe 100, here the outer casing 511 of timing unit 510 is reversibly attached to syringe top end 103 by a foam disk 548 having an adhesive coating on each side. The foam disk 548 is materially consistent with 3M™ double-sided bonding tapes and similarly constructed products. The syringe top end 103 and the side of the outer casing 511 on which battery cover 119 is located, should be pressed against opposite sides of the foam disk 548 to engage the adhesive coating of each side. Once assembled, the syringe 500 can be used.

In another embodiment not seen in the drawings, the plunger 101 is formed directly into the outer casing 111. In this embodiment, the plunger top end is permanently attached to the outer casing, and cannot be removed by the user.

In another embodiment not seen in the drawings, the timing unit 110 is connected to the barrel 105 rather than the plunger top end 103. In this embodiment, care is taken to not obstruct the movement of the plunger 101 relative to the barrel 105.

Although the invention has been described and illustrated with a certain degree of detail or with reference to one or more particular embodiments, it is understood that the present disclosure has been made only by way of example. It should be understood that the invention is not intended to be limited to the particular forms disclosed. Furthermore, the invention is amenable to various modifications and alternative forms. Obvious variations and other various changes in the composition, combination, and arrangement of parts can be utilized to by those skilled in the art without departing from the spirit and scope of the invention, as herein disclosed and claimed.

1. A syringe comprised of:
a plunger, having a top end, a plunger tip, and an elongated member connecting the two;
a barrel, surrounding the plunger tip and having an inner wall that makes a sliding seal with the plunger tip, a barrel flange at one end of the barrel, a barrel tip at the other end of the barrel, and at least one unit marking along the length of the barrel; and
a timing unit, having an outer casing, at least one input button, at least one display, at least one audio indicator, at least one microprocessor, at least one memory unit, and a power source.

2. The syringe of claim 1, where the timing unit is further comprised of a visual alarm indicator.

3. The syringe of claim 1, where the timing unit is further comprised of a power sensor in communication with the microprocessor.

4. The syringe of claim 1, where the power source is comprised of at least one battery positioned inside of the outer casing.

5. The syringe of claim 1, where the timing unit is attached to the top end of the plunger.

6. The syringe of claim 1, where the timing unit is reversibly attached to the top end of the plunger by a strap connected to the outer casing.

7. The syringe of claim 1, where the timing unit is reversibly attached to the top end of the plunger by a clamp connected to the outer casing.

8. The syringe of claim 1, where the timing unit is reversibly attached to the top end of the plunger by a threaded flange that encompasses the plunger top end and engages a threaded ring connected to the outer casing.

9. The syringe of claim 1, where the timing unit is reversibly attached to the top end of the plunger by a set of magnets.

10. The syringe of claim 1, where the timing unit is reversibly attached to the top end of the plunger by inserting an adhesive material between the two.

11. The syringe of claim 10, where the adhesive material is a foam disk having an adhesive coating on at least two sides of the foam disk.

12. The syringe of claim 4, where the timing unit is further comprised of:
a visual alarm indicator,
a power sensor, communicating information about the battery to the at least one microprocessor;
a first button, labeled “start/stop”;
a second button, labeled “set”;
a third button, in the shape of an up arrow;
a fourth button, in the shape of a down arrow; and
a removable battery cover panel built into the outer casing.

13. The syringe of claim 12, where the timing unit is attached to the top end of the plunger.

14. The syringe of claim 12, where the timing unit is reversibly attached to the top end of the plunger by a strap.

15. The syringe of claim 12, where the timing unit is reversibly attached to the top end of the plunger by a clamp.

16. The syringe of claim 12, where the timing unit is reversibly attached to the top end of the plunger by a threaded flange that encompasses the plunger top end.

17. The syringe of claim 12, where the timing unit is reversibly attached to the top end of the plunger by a set of magnets.

18. The syringe of claim 12, where the timing unit is reversibly attached to the top end of the plunger by inserting an adhesive material between the two.

19. The syringe of claim 18, where the adhesive material is a foam disk having an adhesive coating.

20. The method of using a syringe having an attached timing unit to schedule dispensing liquids.
